(19) United States
(12) Patent Application Publication
(10) Pub. No.: US 2008/0200649 A1
(43) Pub. Date: Aug. 21, 2008

(54) Method

(75) Inventors: Srinivasan Vijayasarathy, Bangalore (IN); Santanu Datta, Bangalore (IN)

Correspondence Address: ASTRAZENECA R&D BOSTON, 35 GATEHOUSE DRIVE, WALTHAM, MA, 02451-1215, US

(73) Assignee: AstraZeneca AB, Sodertalje (SE)

(21) Appl. No.: 12/070,862

(22) Filed: Feb. 21, 2008

Related U.S. Application Data
(63) Continuation of application No. 10/875,066, filed on Jun. 22, 2004, now abandoned.
(60) Provisional application No. 60/480,655, filed on Jun. 23, 2003.

(51) Int. Cl. C07K 14/00

(52) U.S. Cl. 530/350

(57) ABSTRACT

A method for optimised refolding of a protein which method comprises selecting for the protein at least three control factors which affect refolding of the protein and at least three levels for each control factor, making a Taguchi matrix of the control factors and levels, conducting experiments as required by the matrix, and refolding the protein in a process using the control factors at levels determined by the outcomes of the matrix experiments.

The present invention relates to the design and optimisation of protein refolding. In particular the invention relates to the use of such optimised proteins in drug discovery processes.

Refolding of recombinant protein is of key importance in the field of Biotechnology. It is a multi-factorial, inter-dependent dynamic process. Several problems are encountered during the refolding process, the primary being the tendency of denatured protein to aggregate in the refolding buffer. This is mainly due to:
The protein concentration, which is directly proportional to the multi-molecular interactions
The temperature, which controls the hydrophobic interactions. Denatured states and partially folded proteins have exposed hydrophobic surfaces, and so can aggregate, interfering with complete folding
The residual denaturant concentration, which influences the folded vs. denatured states
The pH influencing the local ionisation and charge distribution of the refolding protein

Recombinant proteins over-expressed in Escherichia coli are often accumulated as insoluble particles called inclusion bodies. Since proteins in inclusion bodies are usually inactive, they must be solubilized by a denaturing agent such as 8 mol/L urea or 6 mol/L guanidine HCl and refolded to recover their native steric structures having biological activities. A solubilized protein solution is usually added into a large volume of a refolding buffer in order to reduce the concentration of a denaturing agent and also to avoid aggregate formation of protein molecules in the course of renaturation

It is desirable to obtain a fully active protein in a state as similar to the wild-type state or to the native conformation. In the process of drug discovery, stable enzymes are required for high throughput screening (HTS). The more stable the enzyme the greater is the efficiency of compound screening.

Any multi-factorial process requires optimization. Any attempts that have been made at the optimization of protein refolding have been done by the COST method. The COST method is the method of Changing One Separate factor at a Time. This method rarely leads to improvement of a complicated process, but very often leads to the wrong conclusion that the process is running at its optimum. The reason being, COST is unable to identify interactions among the factors that influence the process. Another undesirable feature of the COST method is that it results in several protein folding protocols, each of which, though active, yield proteins varying in quality. In addition, this method of optimizing one variable at a time is extremely cumbersome, requiring a large amount of experimental work, time and money.

We have now devised an improved method for the design and optimisation of protein refolding. This method is based on the Taguchi Method of Optimisation which was developed in the quite separate field of product engineering by Dr. Genichi Taguchi, as a method of improving engineering productivity. Also called the Robust Design Method, the Taguchi technique places a great deal of importance on the reduction in variability of products and processes. The true power of Taguchi methods comes from their simplicity of implementation. We have applied Taguchi principles to the factors involved in protein refolding so as to optimise the protein so obtained. We have observed that protein refolded by the Taguchi method is several times more stable than the same protein refolded by traditional methods.

Therefore in a first aspect of the invention we provide a method for optimised refolding of a protein which method comprises selecting for the protein at least three control factors which affect refolding of the protein and at least three levels for each control factor, making a Taguchi matrix of the control factors and levels, conducting experiments as required by the matrix, and refolding the protein in a process using the control factors at levels determined by the outcomes of the matrix experiments

By “optimised” we mean the best combination of levels as determined by the experiments conducted according to the Taguchi matrix.

The Taguchi Loss Function is conveniently applied to determine the best signal to noise ratio for each control factor. The relevant formula is

[symbol]

### Where,

SNL=signal-to-noise ratio

n=number of components

y=product yield

The Loss Function measures quality. It establishes a measure of the products' deviation from the target value. Measuring loss encourages a focus on achieving less variation.

Taguchi defines loss as a quadratic expression in terms of measured quality characteristics of the part/variant that ranges between the target value and the specifications limits, that is upper and lower specification limits. The loss function is defined such that when the part/variant is made on the target, the loss is absent.

The signal-to-noise (S/N) ratio is simply a logarithmic transformation version of Mean Standard Deviation. (MSD—Mean squared Deviation is a number (no units) representing the average deviation of the results from the target, or the average in the absence of a target, and is strictly a function of the average and standard deviation). Thus, the S/N ratio is the same as the MSD of the data set plotted in a log (to the base 10) scale with a −10 multiplier. The negative multiplier changes the desirability from smaller is better for MSD to bigger is better for the S/N ratio. Thus the S/N ratio represents the status of performance with respect to the variation and a high S/N ratio means that there is high sensitivity with least error.

For each component, the optimal condition is that which gives the largest SNL. [symbol]
SNL [symbol]
a

From the graph we see that variant at level ‘a’ will be the most optimum condition (since SNL is highest at point. ‘a’).

Convenient control factors for a protein include:
The protein concentration, which is directly proportional to multi-molecular reactions.
The temperature, which controls the hydrophobic interactions. Denatured states and partially folded proteins have exposed hydrophobic surfaces, and so can aggregate, interfering with complete folding
The residual denaturant concentration, which influences the folded versus denatured states
The pH influencing the local ionisation and charge distribution of the refolding protein

The three or more levels for each control factor are conveniently selected to cover an appropriate range for the particular factor. If required, four or more levels may be selected. Further examples include five or more, six or more, or seven or more levels.

The protein to be refolded is conveniently an enzyme, for example a kinase, protease, or polymerase. Further proteins include those with defined quantifiable function (non-enzymatic functions) such as receptor proteins binding to ligands, repressor and activator proteins which bind to biological macromolecules. Any protein whose function can be monitored under a defined set of conditions may be used.

The protein is conveniently a recombinant protein. However, the method is equally applicable for all proteins including wild-type (isolated and purified directly from source organisms) proteins. The method provides opportunities for exploring uncharacterised functions of any protein. In particular the method allows you to impart to a refolded protein desired characteristics such as, for example, altered/alternate substrate and ligand binding specificities, thermal stability/unstability and desired pH range for optimal activity. Once such a protein has been isolated it may be used as a template to genetically engineer the required folds in a structurally related protein to obtain same activity. By structurally related we mean for example proteins that share a common genetic origin or simply common structural features.

Therefore in a further aspect of the invention we provide the use of a method of the invention to engineer a refolded protein having non wild-type characteristics.

Particular proteins for use in the above aspect include enzymes or receptors which are promiscuous or degenerate in terms of their specificity in accommodating multiple substrates or ligands. These include protein transferases and biogenic amine receptors. By way of non-limiting example the human and malarial HGPRT proteins are structurally related but the malarial enzyme recognises guanine, hypoxanthine and xanthine whereas the mammalian enzyme does not recognise xanthine. Therefore, by protein refolding, either xanthine specificity may be imparted to the human protein or xanthine specificity may be removed from the malarial enzyme. The resulting crystal structures reveal those critical features involved in this transformation.

Similar possibilities with other enzymes such as phenol sulfotransferase, belonging to sulfotransferase (ST) family which transfers a sulfuryl group from a common sulfonate donor like 3′-phosphoadenosine 5′-phosphosulfate (PAPS) to a nucleophilic acceptor. Phenol sulfotransferase, in addition to PAPS can utilize other nucleotides as substrates although less effectively. By site directed mutagenesis, Hsiao, Y S and Yang, Y S (Biochemistry 2002, October 29; 41(43): 12959-66) have shown that the nucleotide specificity of phenol sulfotransferase can be changed from PAP to AMP. By varying refolding conditions it is possible to alter substrate specificity from PAP to AMP.

Another example is M.tb glycine and alanine dehydrogenases. It has shown that the enzyme glycine dehydrogenase showed the glyoxylate amination but failed to exhibit glycine deamination activity. This work is reported in Can. J. Microbiol. (2002) Jan: 48(1) 7-13. Again, it is possible to impart the reverse reaction, by altering and optimising refolding conditions such that the specificity is reversed quantitatively. Significantly the altered specificity is from the product(s) and the enzyme makes the original substrate.

The refolded protein is conveniently for use in drug discovery, for example in an assay such as a screen, particularly a high throughput screen.

Where the protein is an enzyme, the quality of this is preferably determined by applying the Selwyn Test. This determines whether a decrease in the rate of a reaction is due to inactivation of the enzyme. For a reaction in which all the parameters except the enzyme concentration are kept constant, plots of formed product against the abscissa of time multiplied by enzyme concentration should be superimposable.

However, when the enzyme gets denatured or deactivated during the course of a reaction, the concentration of the enzyme itself becomes a time-dependent quantity. As a result, observations for different concentrations of the enzyme fall on different curves. Thus, depending on the curves obtained, we can determine the stability, and thus the quality of the enzyme. The Selwyn Test is further described and illustrated in the specific description hereinafter.

The person of ordinary skill will be able to select convenient reaction components and conditions for protein refolding.

Convenient additives include reducing or oxidizing agents, zwitterionic compounds, detergents, stabilizing agents (such as arginine, glycerol etc), salts, cofactors and proteins (for example in case of multimers that are heteromeric and wherein concurrent refolding towards a desired reconstitution/association into the functional heteromer of the different, individual monomers may be accomplished).

Convenient temperatures include, in solution phase reactions, Zero degree centigrade to 45 degree centigrade and for proteins of thermophilic organisms up to 100 degree centigrade.

Any convenient pH may be used ie. PH 1 to 14. Convenient protein concentration may be in the range 1 microgram to 1000 micrograms.

The residual denaturant concentration is conveniently one third to one 200^{th} of the undiluted stock concentration.

Salt concentration is conveniently up to 4 Molar.
Cofactor concentrations are conveniently up to 4 Molar.
Additive concentrations are conveniently up to 5 Molar.

In a further aspect of the invention we provide a refolded protein prepared according to the method of the invention and any aspect thereof. We further provide the use of such protein in a drug screening assay.

The invention will now be illustrated but not limited by reference to the following specific description, example, tables and figures wherein:

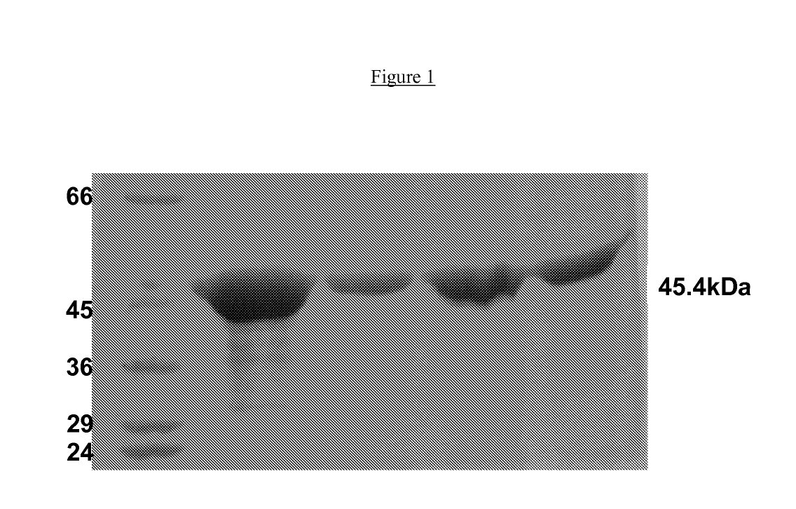

FIG. 1 shows an SDS-PAGE gel run, before assay, at 150V to analyse the isolated isocitrate dehydrogenase protein (band shown at 45.5 kDa).

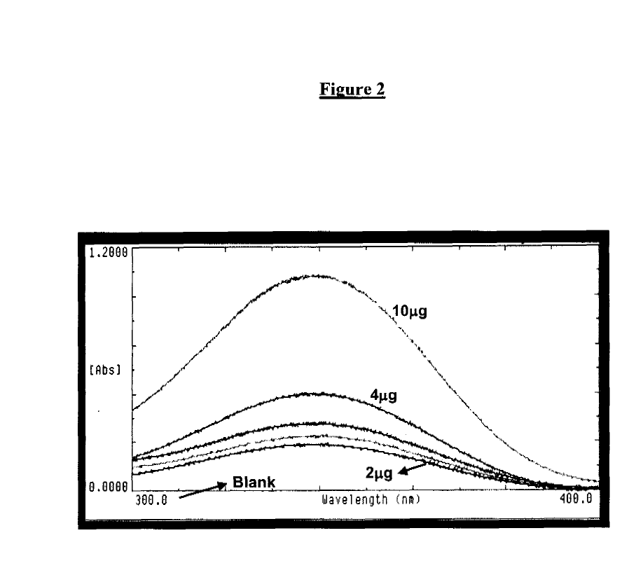

FIG. 2 shows the spectrum obtained from the isocitrate dehydrogenase assay by spectrophometric method (340 nm). As seen, the samples show an absorption maximum at 340 nm.

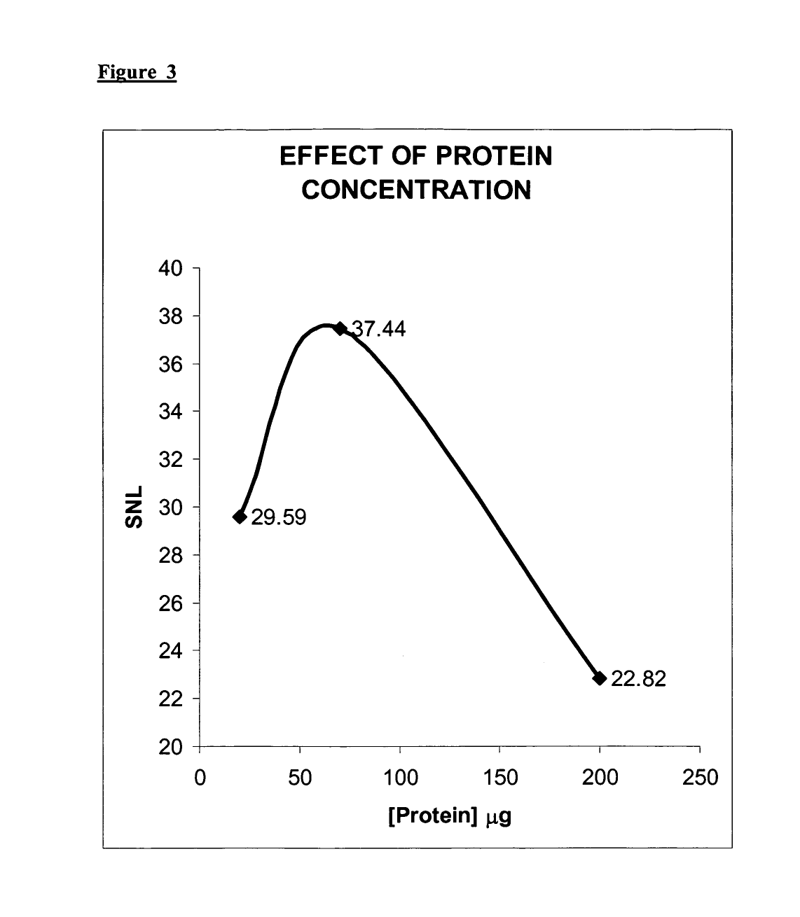

FIG. 3 shows the effect of protein concentration on signal/noise levels for the isocitrate dehydrogenase protein from M. tb.

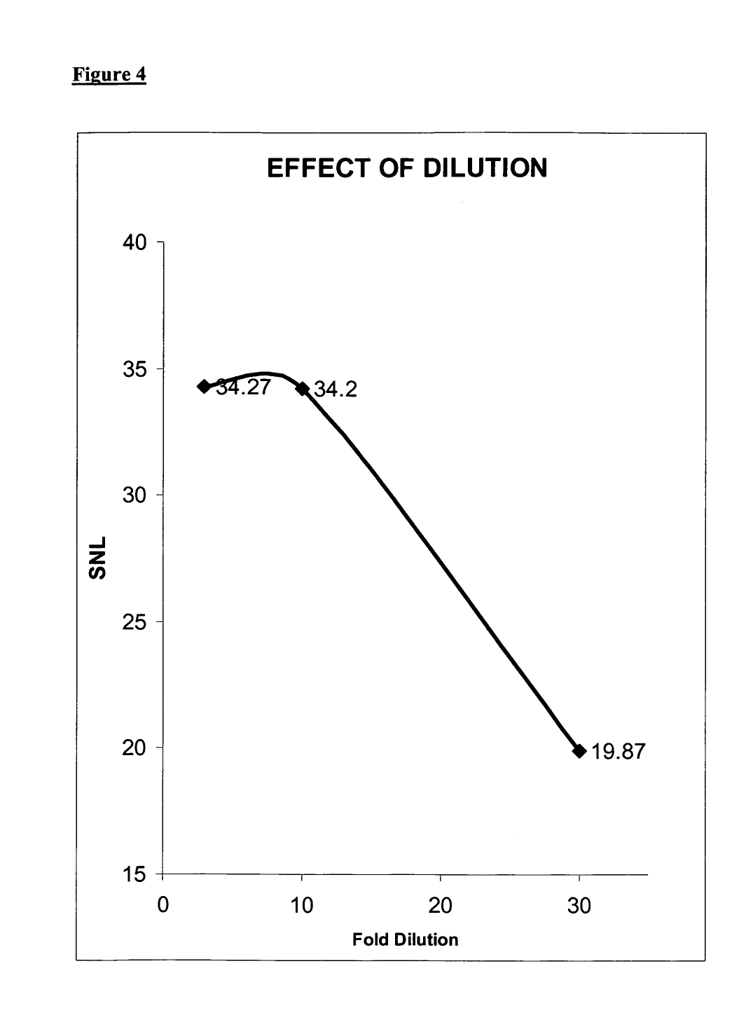

FIG. 4 shows the effect of dilution on signal/noise levels for the isocitrate dehydrogenase protein from M. tb.

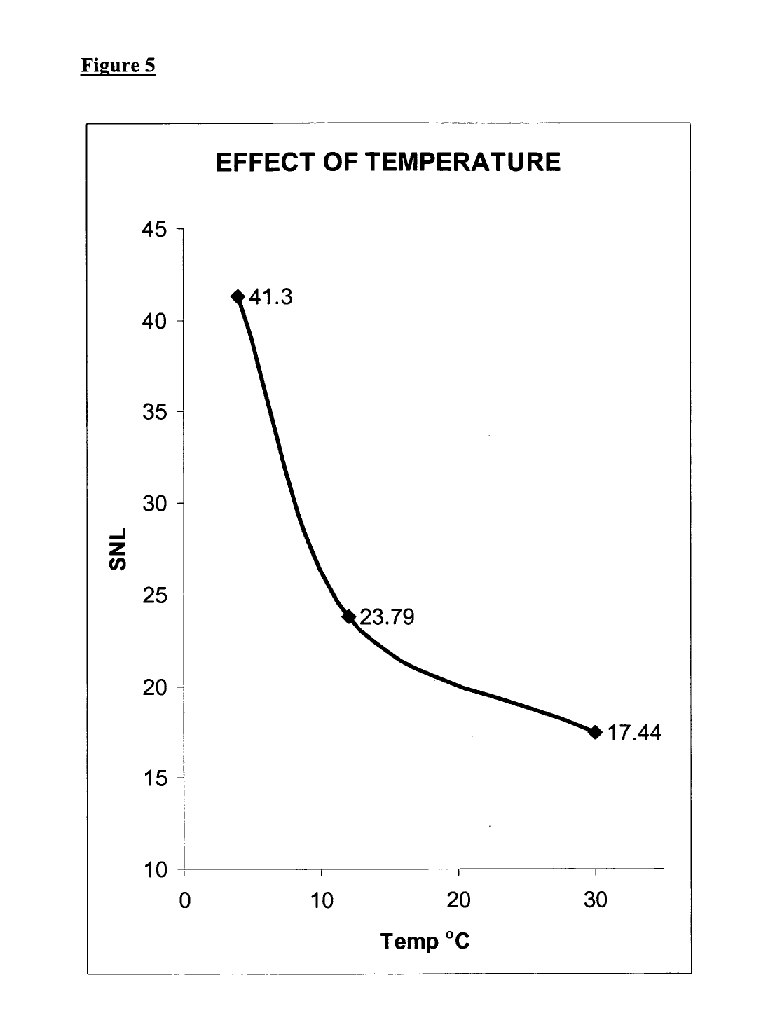

FIG. 5 shows the effect of temperature on signal/noise levels for the isocitrate dehydrogenase protein from M. tb.

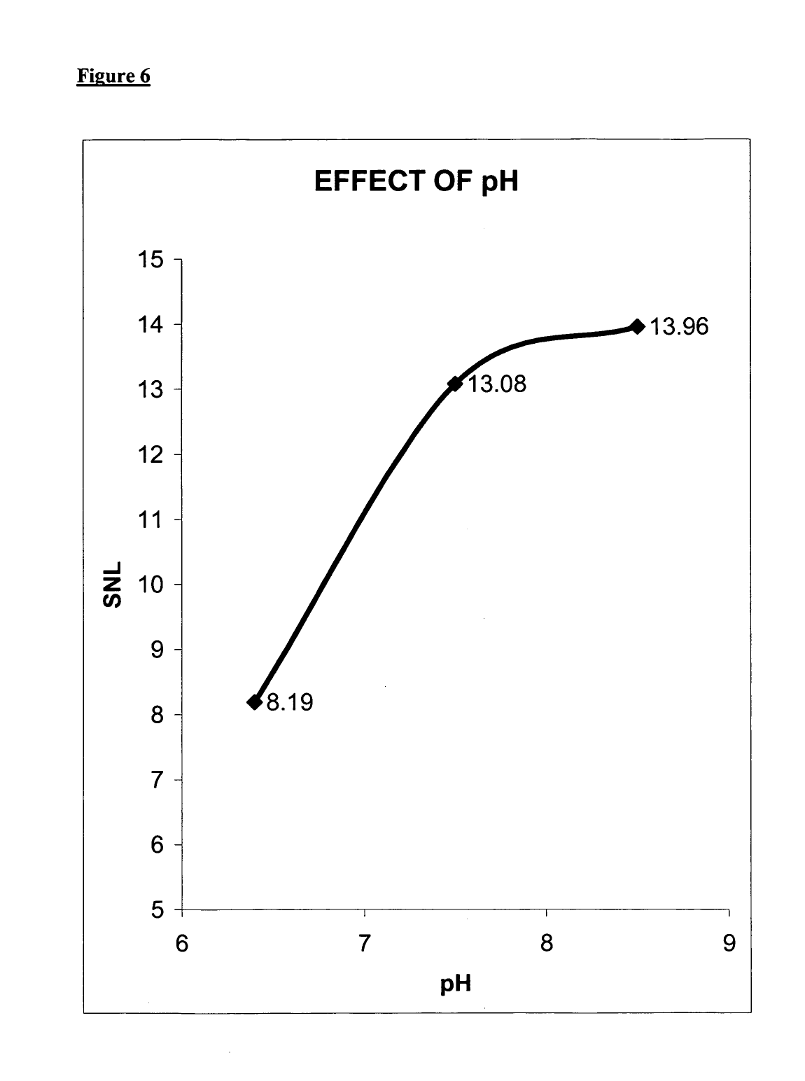

FIG. 6 shows the effect of pH on signal/noise levels for the isocitrate dehydrogenase protein from M. tb.

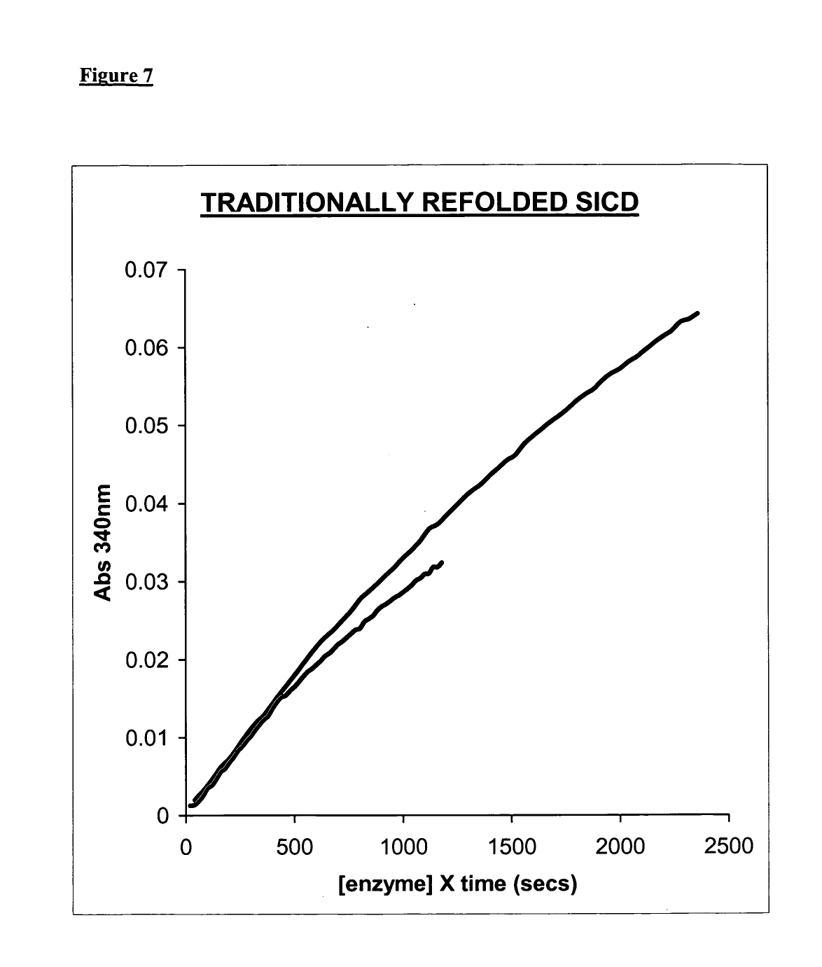

FIG. 7 shows the results of the Selwyn test used to test the quality of a traditionally refolded isocitrate dehydrogenase protein from M.tb. It can be seen that the enzyme is not very stable as it begins to be denatured/inactivated in approximately 600 seconds.

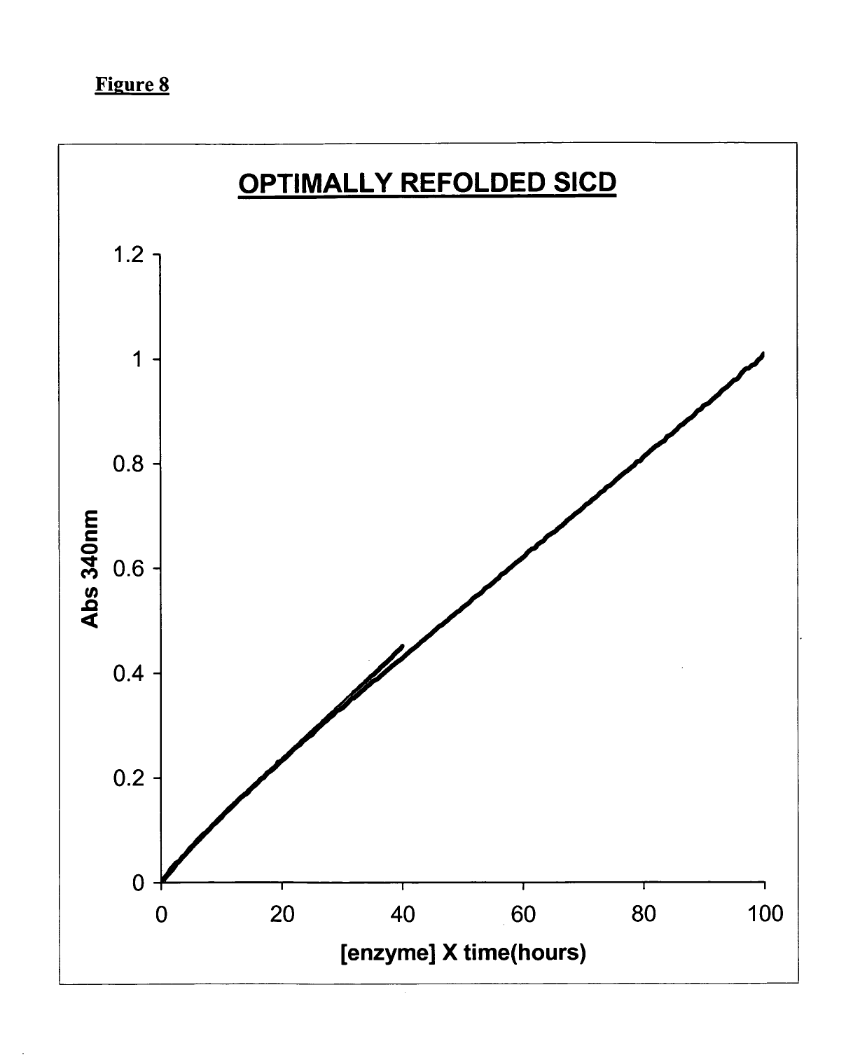

FIG. 8 shows the results of the Selwyn test used to test the quality of isocitrate dehydrogenase from M.tb as optimally refolded using the method of the invention. The enzyme is not denatured or inactivated for several hours.

### SPECIFIC DESCRIPTION

A robust process design is one that does not change with changing noise. Variables, like ambient temperature, humidity, changes to machines and operators and raw material variation, that affect product quality but are beyond our control, are referred to as noise. In other words, the Taguchi Method aims at making products and processes more robust and less susceptible to changes due to outside influences. It includes a set of tables that enable main variations and interactions to be investigated in a minimum number of trials.

Rather than tightening up on the process variables, it is often better to try to adjust the level of these variables to reduce the effect of noise. This way we end up with a product or process that is not only high in quality, but gives us consistently high quality.

The traditional optimization of a process involving 4 reaction components, using three different levels for each, would require a total of 3⁴=81 trials. This being very time-consuming and expensive, all 81 trials are never carried out. A few sets of experiments are carried out and the one yielding best results is taken to be the optimum, though it might not actually be. In the case of the Taguchi method, the same process can be optimized with just 9 trials!! The equation used is:

[symbol]

Where, E=the number of experiments

k=the number of components to be tested

E has to be a multiple of three, if not the number of experiments is taken to be the next multiple of three.

Taguchi proposed an orthogonal array to systematically vary and test the different levels of the control factors considered. For a process involving 4 components (1, 2, 3, 4) using three different levels (A, B, C) for each, the orthogonal array is as follows:

The Taguchi Matrix:

| | 1 | 2 | 3 | 4 |
|---|---|---|---|---|
| Experiment 1 | A | A | A | A |
| Experiment 2 | A | B | B | B |
| Experiment 3 | A | C | C | C |
| Experiment 4 | B | A | B | C |
| Experiment 5 | B | B | C | A |
| Experiment 6 | B | C | A | B |
| Experiment 7 | C | A | C | B |
| Experiment 8 | C | B | A | C |
| Experiment 9 | C | C | B | A |

The properties of the array are such that between each pair of columns, each combination of levels occurs the same number of times.

To estimate the effect of the individual components, the Taguchi Loss Function is used:

[symbol]

### Where,

SNL=signal-to-noise ratio

n=number of components

y=product yield

The Loss Function measures quality. It establishes a measure of the products' deviation from the target value. Measuring loss encourages a focus on achieving less variation. As we understand how even a little variation from the nominal results in a loss, the tendency would be to try and keep product and process as close to the nominal value as possible. This is what is so beneficial about the Taguchi loss. It always keeps our focus on the need to continually improve.

For each component, the optimal condition is that which gives the largest SNL. [symbol]
SNL
   a

From the graph we see that variant at level ‘a’ will be the most optimum condition (since SNL is highest at point. ‘a’).

### The Selwyn Test and the Selwyn Progress Curves:

An indication of the quality of an enzyme is its stability. In order to determine whether a decrease in the rate of a reaction is due to inactivation of the enzyme, we perform a simple test called the Selwyn Test. The curves plotted are the Selwyn Progress Curves.

### Where,

[E]=total initial enzyme concentration

t=time

[P]=product concentration

Michaelis and Davidson proposed the above relation in 1911. As per this equation, for a reaction in which all the parameters except the enzyme concentration are kept constant, plots of formed product against the abscissa of time multiplied by enzyme concentration should be super-imposable.

However, when the enzyme gets denatured or deactivated during the course of a reaction, the concentration of the enzyme [E], itself becomes a time-dependent quantity. As a result, observations for different concentrations of the enzyme fall on different curves.

Thus, depending on the curves obtained, we can determine the stability, and thus the quality of the enzyme.

### EXAMPLE

### Materials and Methods

Materials: All chemicals used were obtained from Sigma Chemical Company, St. Louis, Mo., and were of the highest purity. The Non-Detergent SulfoBetaine (NDSB) was obtained from Calbiochem Corporation.

Organisms and Growth Conditions: Experiments were conducted with the BL21DE3 strain of E. coli cells. The media used was the Luria Bertani broth, containing 1% (M/V) tryptone peptone, 0.5% (M/V) yeast extract, 1% (M/V) sodium chloride and 1.5% (M/V) bacto-agar. The E. coli cells were grown in the above medium with ampicillin (100 micrograms/ml).

Preparation of Competent Cells: The BL21DE3 E. coli cells were grown in the LB broth (with ampicillin) up to an OD of 0.8 (Absorbance at 600 nm). The cells were then spun down at 7000 RPM for 10 minutes. The supernatant was discarded and the pellet was washed twice with ice-cold Milli-Q water. Following this, one washing was carried out with 10% glycerol. The cell pellet was then suspended in 10% glycerol. 0.1% (V/V) of 1 mM HEPES (pH 7) was added to the solution. The cell extract was then frozen in liquid nitrogen and stored at −70° C.

Cloning and Expression of the ICD protein: The M tb ICD (Rv3339c) was expressed in E. coli using a pET8c vector.

Purification of the ICD Protein: Cell lysates were prepared by re-suspending the frozen cell pellets in 15 ml of sonication buffer. The sonication buffer was made up of 50 mM Tris (pH 8), 1 mM DTT, 1 mM EDTA and 0.15M potassium chloride. Sonication (70%) was carried out for the sample for 5 minutes, until the solution starts to become clear. The OD of each sample was then taken and was found to have decreased to one-fifth the OD of the samples before sonication. The samples were then spun at 7000 RPM for 10 minutes. The pellet thus obtained was dispersed in a buffer containing 10 mM Tris (pH 8.0), 1 mM EDTA and 1 mM DTT.

The samples were then loaded on a sucrose gradient. For this 5 ml of 60% sucrose was used, over which 5 ml of 15% sucrose was added and finally 2 ml of the solution was loaded at the top. All the samples were spun at 30000 RPM for 90 minutes, after which the supernatant was discarded. The pellet obtained was washed twice with 1% triton X 100 and then centrifuged at 30000 g for 30 minutes at 4° C. The pellet was then stored at −20° C.

Solubilization of the Protein from Inclusion Bodies: The frozen pellet, obtained after purification of the inclusion bodies, was dissolved in 2 ml solubilizing buffer, and kept at 4° C. for one hour. Solubilizing buffer was made with 50 mM HEPES (pH 7.5), 6M guanidine HCl, 25 mM DTT. Insoluble material was then removed by centrifugation at 100000 g for 10 minutes.

Taguchi Optimization of Experimental Parameters: Parameters of protein refolding that were taken into consideration for this investigation were 1) pH, 2) temperature, 3) protein concentration and 4) dilution (residual guanidine concentration). The variables used were as follows:

| | | | |
|---|---|---|---|
| PH | 6.4 | 7.5 | 8.5 |
| Temperature | 4° C. | 12° C. | 30° C. |
| Dilution (residual | 3-fold | 10-fold | 30-fold |
| guanidine | | | |
| concentration) | | | |
| Final protein | 20 | 70 | 200 |
| Concentration | | | |
| (μg/ml) | | | |

The orthogonal array used was:

| | | Temp | | Protein |
|---|---|---|---|---|
| Concentration | pH | ° C. | Dilution | (μg) |
| Experiment 1 | 6.4 | 4 | 3 | 20 |
| Experiment 2 | 6.4 | 12 | 10 | 70 |
| Experiment 3 | 6.4 | 30 | 30 | 200 |
| Experiment 4 | 7.5 | 4 | 10 | 200 |
| Experiment 5 | 7.5 | 12 | 30 | 20 |
| Experiment 6 | 7.5 | 30 | 3 | 70 |
| Experiment 7 | 8.5 | 4 | 30 | 70 |
| Experiment 8 | 8.5 | 12 | 3 | 200 |
| Experiment 9 | 8.5 | 30 | 10 | 20 |

For use at different temperatures, select buffers were prepared. For use at pH 6.4, 100 ml of 500 mM PIPES (pKa=7.1), 200 mM KCl buffer was prepared by dissolving 15.119 g of PIPES free acid in 90 ml of pure water. The solution was prepared at the laboratory temperature of 25° C. Thus, for use at 4° C., 12° C. and 30° C., the PIPES buffer was adjusted to pH of 6.22, 6.28 and 6.44 respectively. The volume was finally adjusted to 100 ml with water.

Similarly, for use at pH 7.5, 100 ml of 500 mM HEPES (pKa=7.66), 200 mM KCl buffer was prepared by dissolving 11.915 g of HEPES free acid in 90 ml pure water. Since the temperature of preparation of the buffer was 25° C., for use at 4° C., 12° C. and 30° C., the HEPES buffer was adjusted to pH of 7.2, 7.31 and 7.56 respectively. The volume was then adjusted to 100 ml with water.

For use at pH 8.5, 100 ml of TAPS (pKa=8.51), 200 mM KCl buffer was prepared by dissolving 12.615 g of TAPS free acid in 90 ml of pure water. Once again, for use at 4° C., 12° C. and 30° C., the TAPS buffer was titrated to pH of 8.08, 8.24 and 8.6 respectively. The volume, again, was made up to 100 ml with water.

All buffers had a final concentration of 1M NDSB-201.

On completion of the Taguchi method, the protein solutions obtained were dialysed against 0.5×PBS containing 10% glycerol. This was followed by concentration of the solutions by ultra-filtration through micron filter.

Solutions thus obtained were quantified for protein content and then used for ICD assay.

Estimation of Protein Concentration: The Coomassie Reagent Protein Assay was used to determine the protein concentration of the solution obtained after the re-suspension of the inclusion body pellet. For this assay the protocol followed was the Standard Microplate Protocol. The working range is 100-1500 μg/ml. 10 ml of each sample was pipetted out into individual wells. 300 μl of the Coomassie Plus Reagent was then added to each well. The plate was mixed on a plate shaker for 30 seconds. The absorbance was measured at 595 nm. BSA samples of varying concentrations were used as the standards.

Assay of the ICD Protein: ICD is an enzyme of the TCA cycle. It converts isocitrate to α-ketoglutarate, with the liberation of carbon dioxide. It simultaneously causes the reduction of NADP to NADPH. This NADPH has an absorption maximum at 340 nm. Thus, the assay of the ICD protein was carried out by spectrophotometric method at the wavelength of 340 nm. The reduction of NADP to NADPH was taken as a measure of the protein activity

Electrophoresis: An SDS-PAGE gel was run, before assay, at 150V to analyse the isolated protein. The gel was run with a 10% resolving buffer and a 5% stacking buffer. The results are shown in FIG. 1.

Product Characterization This is the spectrum obtained from ICD assay by spectrophotometric method (340 nm). As seen in FIG. 2, the samples show an absorption maximum at 340 nM.

### Results

On completion of the Taguchi method, 4 graphs were generated, showing the optimal conditions of protein concentration, dilution, temperature and pH for refolding of M. tb ICD protein.

FIG. 3 shows the effect of protein concentration, we see that the optimum protein concentration for M tb ICD refolding is 50-70 μg.

FIG. 4 shows the effect of dilution. From this graph we deduce that a 1:10 fold dilution is optimal for M. tb ICD refolding.

FIG. 5 shows the effect of temperature. We deduce from this graph that a temperature of 4° C. is the optimum for this protein.

FIG. 6 shows the effect of temperature. We deduce from this graph that the optimum pH range for refolding of M. tb ICD is 7.5-8.5.

### Stability Studies

The Selwyn test was used to test the quality of the protein, details of which, are given below.

FIG. 7 shows that for a traditionally folded M tb ICD, the enzyme is not very stable as it begins to get denatured/inactivated in approximately 600 seconds.

FIG. 8 shows that M. tb ICD optimally refolded by Taguchi Method of Optimisation is much more stable. The enzyme does not get denatured or inactivated for several hours.

### Discussion

Protein refolding is a vital process in the utility of proteins in Biotechnology. Proteins expressed in E. coli often accumulate as insoluble inclusion bodies, and therefore solubilization and renaturation of these proteins is of utmost importance. This is necessary in order to obtain the fully active proteins in a state as similar to the wild state or the native conformation.

Protein refolding protocols are still being developed one-by-one, by the optimization of one parameter at a time. Thus, there are several protein folding protocols available, each giving a protein which, though active, is of varying quality. We need to choose the right procedure that will allow renaturation of recombinant proteins deposited in inclusion bodies, giving high yields. To our knowledge, this is the first attempt at refining and optimising the most critical key parameters involved in refolding, at the same time, to arrive at the precise conditions that give the best product.

We have employed the industrial Taguchi methodology for the refolding of M. tb ICD protein, applying the Taguchi Matrix for four reaction components, considering three concentration levels for each. This Taguchi Method of Optimization enables us to define the precise conditions, for each of the parameters, which give optimally folded or improved quality protein.

When the protein obtained after this method of refolding is compared with traditionally refolded protein, we see a striking difference in the quality of the protein in terms of stability. This was observed by the Selwyn Progress curves.

In the process of drug discovery, stable enzymes are required for HTS (High Throughput Screening). More stable the enzyme greater is the efficiency of compound screening. Proteins refolded by the Taguchi method have been seen to be several times more stable than the same protein refolded by traditional methods. This leads us to believe that this method has important implications in the robustness of assays.

In conclusion, this refolding of Mycobacterium tuberculosis isocitric dehydrogenase by the Taguchi Method of Optimization is a case study. The optimum for other proteins would certainly vary, with their own individual characteristic conditions for optimal refolding. The major thrust of this work is that this methodology provides the most amenable handle to choose the precise set of conditions for optimal renaturation, through a refinement process, from a known window of a reasonable range that is preset for each of the variables. Similar methods should be adopted in other fields of scientific research to ensure complete optimization of processes and attainment of superior quality product.

### REFERENCES

1. Misawa, S., Kumagai, I. Refolding of Therapeutic Proteins Produced in Escherichia coli as Inclusion Bodies, Biopolymers (Peptide Science), 1999, Vol. 51, 297-307
2. Selwyn, M. J., A Simple Test for Inactivation of an Enzyme During Assay, Biochim Biophys Acta, 1965 Jul. 29; 105(1):193-5
3. Vuillard, L., Rabilloud, T., Goldberg, M. E., Interactions of Non-Detergent Sulfobetaines with Early Folding Intermediates Facilitate In Vitro Protein Renaturation, Eur. J. Biochem., 1998, Vol 256, 128-135
4. Dhariwal, K. R., Venkitasubramanium, T. A., NADP-Specific Isocitrate Dehydrogenase of Mycobacterium phlei ATCC 354: Purification and Characterization, Journal of General Microbiology, 1987, Vol. 133, 2457-2460.
5. Lamelli, U. K., Nature, 1970, Vol. 227, 680
6. Cobb, B. D., Clarkson, J. M., A Simple Procedure for Optimising the Polymerase Chain Reaction (PCR) using modified Taguchi Methods, Nucleic Acids Research. 1994, Vol. 22, No. 18, 3801-3805
7. http://www.orszulik.free-online.co.uk, Experimental Design and Taguchi
8. Design of Experiments using the Taguchi Approach-16 steps to product and process improvement. Ranjit K Roy 2001. John Wiley & sons, inc

1. A method for optimised refolding of a protein which method comprises selecting for the protein at least three control factors which affect refolding of the protein and at least three levels for each control factor, making a Taguchi matrix of the control factors and levels, conducting experiments as required by the matrix, and refolding the protein in a process using the control factors at levels determined by the outcomes of the matrix experiments.

2. A method as claimed in claim 1 and wherein the protein is a recombinant protein.

3. A method as claimed in claim 1 or claim 2 and wherein the protein is an enzyme.

4. A method as claimed in claim 1 and wherein the control factors are selected from pH, temperature, protein concentration and residual denaturant concentration.

5. A method as claimed in any previous claim and wherein at least four control factors are selected.

6. A method as claimed in any previous claim and wherein at least four levels are selected for each control factor.

7. A refolded protein prepared according to the method of any one of claims 1-3.

8. Use of a refolded protein as claimed in claim 7 in a drug screening assay.

9. Use of a method as claimed in any one of claims 1-7 to engineer a refolded protein having non wild-type characteristics.
